(19) United States
(12) Patent Application Publication
(10) Pub. No.: US 2010/0076216 A1
(43) Pub. Date: Mar. 25, 2010

(54) LIQUID CRYSTAL COMPOUND, OPTICAL ELEMENT, POLARIZING PLATE, IMAGE DISPLAY APPARATUS, AND OPTICAL RECORDING MATERIAL

(75) Inventors: Sadahiro Nakanishi, Osaka (JP); Mitsuru Ueda, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NITTO DENKO CORPORATION, Ibaraki-shi, Osaka (JP)

(21) Appl. No.: 12/443,590

(22) PCT Filed: Jul. 23, 2007
(86) PCT No.: PCT/JP2007/064452

§ 371 Date: Mar. 30, 2009

(30) Foreign Application Priority Data
Oct. 18, 2006 (JP) 2006-284222

(51) Int. Cl. C07C 255/50; G02F 1/13

(52) U.S. Cl. 558/416; 349/194

(57) ABSTRACT

Provided are a liquid crystal compound which can exhibit liquid-crystalline phase even at low temperatures, is excellent in uniform application performance and compatibility with another component, and suppresses inclusion of a polymeric component other than the object component, and an optical element having little defects in appearance, a polarizing plate, an image display apparatus, and an optical recording material using the liquid crystal compound. The liquid crystal compound of the present invention includes two or more chemical structures Q each represented by a general formula (1).

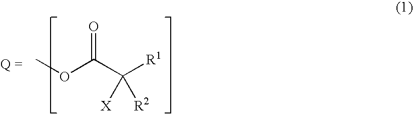

### TECHNICAL FIELD

The present invention relates to a liquid crystal compound and a use thereof. More specifically, the present invention relates to a liquid crystal compound, and an optical element, a polarizing plate, an image display apparatus, and an optical recording material using the liquid crystal compound.

### BACKGROUND ART

For an optical film such as an optical compensation film used for a liquid crystal display, a birefringent film in which a polymer film is subjected to stretching treatment is used from a requirement to the compatibility of improvement of a display quality of a liquid crystal display device and weight reduction of the device.

However, in the birefringent film, an alignment state thereof is broken at temperatures exceeding a glass transition point of the polymer film. Therefore, the birefringent film has a defect in that operating temperature thereof is limited by the glass transition point.

There has been developed a liquid crystal alignment film having higher and more stable alignment state, which cannot be realized by stretching treatment. The liquid crystal alignment film is provided by subjecting a liquid crystal compound having a liquid crystalline polymer or a polymerizable functional group to alignment treatment in order to obtain alignment such as tilt alignment or twist alignment (refer to Patent Documents 1 to 3) .

A method of using the liquid crystalline polymer includes applying a polymer compound solution expressing thermotropic liquid crystallinity onto a substrate subjected to alignment treatment, and heat-treating the resultant at a temperature at which the liquid crystalline polymer exhibits liquid crystallinity, whereby desired alignment is obtained. After the alignment is completed by the above method, the alignment is fixed by maintaining the liquid crystalline polymer in a glass state.

However, in the liquid crystalline polymer, molecular motion is inhibited by entanglement of molecular chains. Therefore, there have been problems of the liquid crystalline polymer having poor solubility to a solvent and having difficulty in producing a uniform liquid crystal alignment film. Further, the liquid crystalline polymer is inferior in compatibility, with other components, and thus, the liquid crystalline polymer needs to undergo synthesis operations such as copolymerization in order to combine functional sites such as a liquid crystal group, a cross-linking group, and a chiral group.

As a method of obtaining a liquid crystal compound having excellent compatibility, a concept, of a vitrified liquid crystal has recently been reported. The liquid crystal compound expressing such a concept contains a plurality of liquid crystal groups at a terminal thereof, and has a structure in which the liquid crystal group and a core portion are linked to each other via a connecting group. The liquid crystal compound having such a structure enables improvements in solubility to some extent and in uniform application performance. The uniform application performance is effectively exhibited in the case where a liquid crystal phase expressed by a liquid crystal material is a nematic liquid crystal phase alone. On the contrary, when the liquid crystal phase contains a phase which is near the crystal such as a smectic phase, the uniform application becomes difficult at the time of applying the liquid crystal material onto the substrate or performing the alignment treatment, due to liquid crystallization or smectic liquid crystallization. Accordingly, in the vitrified liquid crystal, the uniform application performance is maintained by designing a liquid crystal compound having a substituent at side azimuth of the liquid crystal group for a purpose of decreasing crystallinity of the liquid crystal to thereby express solely the nematic liquid crystal phase. However, there has been desired development of a liquid crystal compound in which a liquid crystal compound having a simple-structured liquid crystal group which do not have a substituent at side azimuth thereof can express solely the nematic liquid crystal phase.

In the case of considering introducing a cross-linking group such as a (meth)acrylic group to acrylic polymer liquid crystal compound, a synthesis of a liquid crystal acrylic polymer having the cross-linking group left behind therein may be attempted by copolymerizing a liquid crystal acrylic monomer with a functional acrylic monomer, but the mixture of liquid crystal acrylic monomer and the functional acrylic monomer is insolubilized by gelation, and therefore, the synthesis of the liquid crystal acrylic polymer has been impossible.

In general, a polymer compound such as an acrylic polymer compound has a distribution in a molecular weight thereof. A molecular weight distribution (Mw/Mn) represented by a ratio of a weight average molecular weight (Mw) to a number average molecular weight (Mn) is 1 in the case of a single molecular weight, and Mw/Mn of a polymer compound synthesized by a general radical polymerization is at least 1.2 or more. Thus, it is natural that a general polymer compound includes a component having larger molar weight (polymeric component) with respect to an average molecular weight of the general polymer compound.

When the above-mentioned material containing the polymeric component other than the object component is used to be processed into a film, there may arise a problem such as phase separation. Thus, it is ideally requested that the polymeric component other than the object component be not included in the material.
Patent Document 1: JP 03-28822 A
Patent Document 2: JP 04-55813 A
Patent Document 3: JP 05-27235 A

### DISCLOSURE OF THE INVENTION

### Problems to be Solved by the Invention

It is an object of the present invention to provide a liquid crystal compound which can exhibit liquid crystallinity even at low temperatures, is excellent in uniform application performance and compatibility with another component, and suppresses inclusion of a polymeric component other than the object component, and an optical element having little detects in appearance, a polarizing plate, an image display apparatus, and an optical recording material using the liquid crystal compound.

### Means for Solving the Problems

The inventors of the present invention have intensively studied to solve the above-mentioned problems. As a result, the inventors have found that a vitrified liquid crystal compound having a structure in which a plurality of liquid crystal (meth)acrylic monomers are added to a plurality of compound cores containing cyanoacetate or acetoacetate is a liquid crystal compound which can solve the above-mentioned problems. Further, they have also found that introduction of a cross-linking group, which has been difficult to perform by polymerization of a conventional acrylic monomer, can be performed easily when the vitrified liquid crystal compound is used.

A liquid crystal compound of the present invention includes two or more chemical structures Q each represented by a general formula (1):

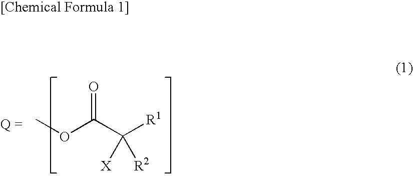

where, in the general formula (1): X represents one of —CN and —COCH₃; and R¹ and R² each independently represent any one of —H, a chemical structure represented by a general formula (2), and a chemical structure represented by any one of general formulae (3a) to (3f):

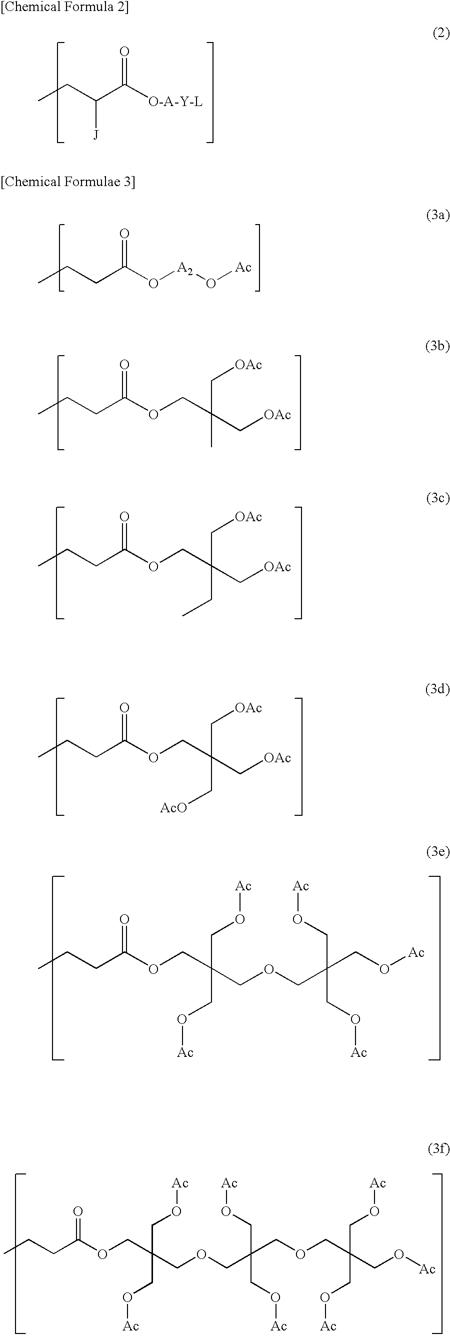

where, in the general formula (2): J represents one of —H and —CH₃; A represents a single bond or an alkylene group having 2 to 12 carbon atoms, in which one —CH₂— or two or more nonadjacent —CH₂—'s present in the alkylene group may be substituted with —O—; Y represents any one of —O—, —COO—, —OCO—, and —OCOO—; and L represents a chemical structure represented by any one of general formulae (4a) to (4g),

where, in the general formulae (3a) to (3f): Ac represents a (meth) acryloyl group; and A₂ represents an alkylene group having 2 to 12 carbon atoms:

[Chemical Formulae 4]

—C_{y}—C_{y} (4a)

—C_{y}—C_{y}—C_{y} (4b)

—C_{y}—Z—C_{y} (4c)

—C_{y}—Z—C_{y}—Z—C_{y} (4d)

—C_{y}—C≡C—C_{y} (4e )

—C_{y}—C≡C—C_{y}—C≡C—C_{y} (4f)

—C_{y}—Z—C_{y}—C≡C—C_{y} (4g )

where, in the general formulae (4a) to (4g): Z represents any one of —COO—, —OCO—, —CONH—, CON(alkyl)-, and —CH═N—; and C_{y}'s each independently represent any one of a phenyl ring, a naphthyl ring, a biphenyl ring, and a cyclohexyl ring which may each have at least one kind of substituent selected from F, CN, an alkoxy group, and an alkyl group.

In a preferred embodiment, the liquid crystal compound includes a chemical structure represented by any one of general formulae (5a) to (5f):

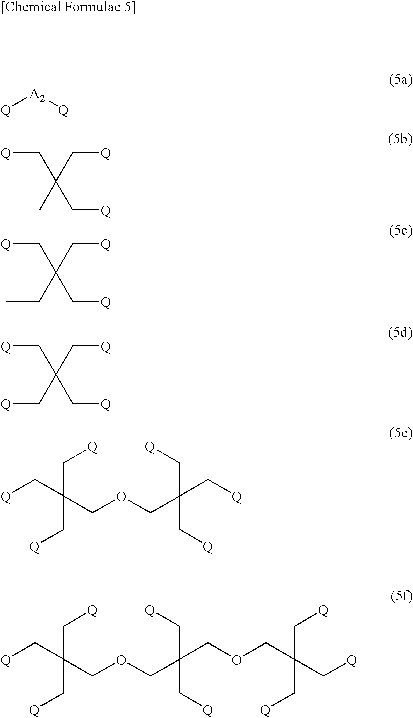

where, in the general formula (5a), A₂ represents an alkylene group having 2 to 12 carbon atoms, in which one —CH₂— or two or more nonadjacent —CH₂—'s present in the alkylene group may be substituted with —O—.

In a preferred embodiment, in the general formula (2) represents —H.

In a preferred embodiment, Y in the general formula (2) represents —O—.

In a preferred embodiment in the general formula (1) represents —CN.

In a preferred embodiment, the liquid crystal compound includes a cross-linkable liquid crystal compound.

In another aspect of the present invention, an optical element is provided. The optical element of the present invention includes the liquid crystal compound of the present invention. Further, the optical element of the present invention includes a cross-linked product formed by cross-linking the liquid crystal compound of the present invention which includes the cross-linkable liquid crystal compound.

In another aspect of the present invention, a polarizing plate is provided. The polarizing plate of the present invention includes the optical element of the present invention.

In another aspect of the present invention, an image display apparatus is provided. The image display apparatus of the present invention includes at least one polarizing plate of the present invention.

In another aspect of the present invention, an optical recording material is provided. The optical recording material of the present invention includes the liquid crystal compound of the present invention.

### EFFECTS OF THE INVENTION

According to the present invention, there can be provided a liquid crystal compound which can exhibit liquid crystallinity even at low temperatures, is excellent in uniform application performance and compatibility with another component, and suppresses inclusion of a polymeric component other than the object component, and an optical element having little defects in appearance, a polarizing plate, an image display apparatus, and an optical recording material using the liquid crystal compound.

### BRIEF DESCRIPTION OF THE DRAWINGS

FIG. 1 is a schematic cross-sectional view of a liquid crystal display apparatus according to a preferred embodiment of the present invention.

FIG. 2 is a mass spectrum of a liquid crystal compound (4).

FIG. 3 is a mass spectrum of a liquid crystal compound (5).

### DESCRIPTION OF REFERENCE NUMERALS

10 liquid crystal cell
11, 11′ glass substrate
12 liquid crystal layer
13 spacer
20, 20′ retardation film
30, 30′ polarizing plate
40 light guide plate
50 light source
60 reflector
100 liquid crystal display apparatus

### BEST MODE FOR CARRYING OUT THE INVENTION

Hereinafter, the present invention is described by way of preferred embodiments of the present invention, but the present invention is not limited to those embodiments. Herein, “(meth)acrylic acid” refers to acrylic acid or methacrylic acid.

### <<Liquid Crystal Compound>>

The polyfunctional compound of the present invention includes two or more chemical structures Q each represented by a general formula (1):

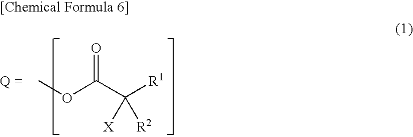

where, in the general formula (1): X represents one of —CN and —COCH₃; R¹ and R² each independently represent any one of —H, a chemical structure represented by a general formula (2), and a chemical structure represented by any one of general formulae (3a) to (3f); and X preferably represents —CN:

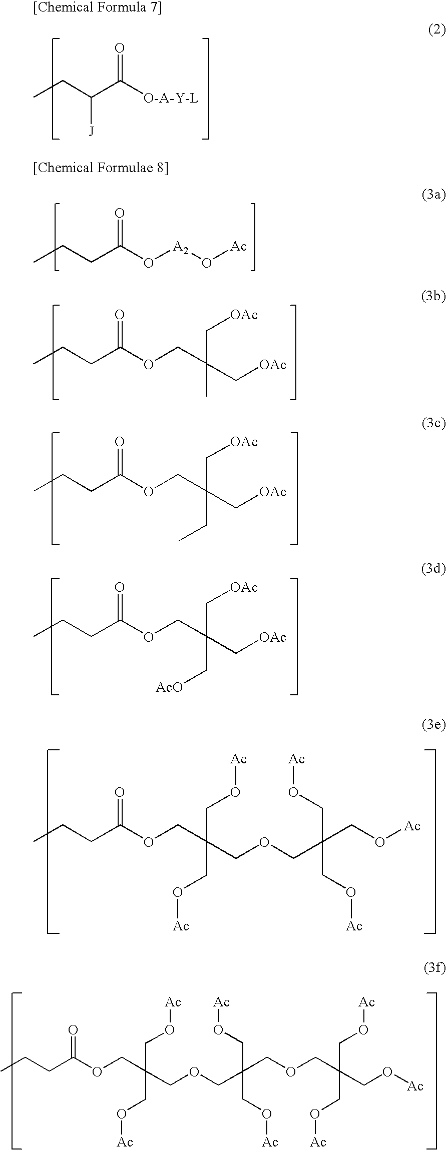

where, in the general formulae (3a) to (3f): Ac represents a (meth) acryloyl group; and A₂ represents an alkylene group having 2 to 12 carbon atoms,

where, in the general formula (2): J represents one of —H and —CH₃; A represents a single bond or an alkylene group having 2 to 12 carbon atoms, in which one —CH₂— or two or more nonadjacent —CH₂—'s present in the alkylene group may be substituted with —O—; Y represents any one of —O—, —COO—, —OCO—, and —OCOO—; L represents a chemical structure represented by any one of general formulae (4a) to (4g); and J preferably represents —H and Y preferably represents —O—:

[Chemical Formulae 9]

—C_{y}—C_{y} (4a)

—C_{y}—C_{y}—C_{y} (4b)

—C_{y}—Z—C_{y} (4c)

—C_{y}—Z—C_{y}—Z—C_{y} (4d)

—C_{y}—C≡C—C_{y} (4e )

—C_{y}—C≡C—C_{y}—C≡C—C_{y} (4f)

—C_{y}—Z—C_{y}—C≡C—C_{y} (4g )

where, in the general formulae (4a) to (4g): Z represents any one of —COO—, —OCO—, —CONH—, CON(alkyl)-, and —CH═N—; and C_{y}'s each independently represent any one of a phenyl ring, a naphthyl ring, a biphenyl ring, and a cyclohexyl ring which may each have at least one kind of substituent selected from F, CN, an alkoxy group, and an alkyl group.

The liquid crystal compound of the present invention includes a chemical structure represented by any one of general formulae (5a) to (5f):

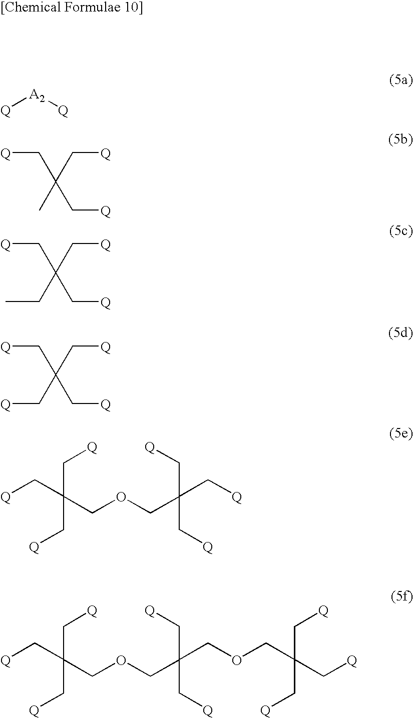

where, in the general formula (5a), A₂ represents an alkylene group having 2 to 12 carbon atoms, in which one —CH₂— or two or more nonadjacent —CH₂—'s present in the alkylene group may be substituted with —O—.

For synthesizing the liquid crystal compound of the present invention, any appropriate method can be employed. The liquid crystal compound is preferably synthesized by: subjecting a raw material compound having an alcohol group to cyanoacetic acid-esterification or acetoacetic acid-esterification to thereby synthesize polyfunctional cyanoacetate or acetoacetate; deprotonating a carbon-hydrogen bond on a carbon sandwiched by a carbonyl carbon of the ester and a carbon of the cyano group or the aceto group and having high acidity; and then substituting the resultant with (meth)acrylate by Michael addition reaction.

Cyanoacetates and acetoacetates each having a carbon sandwiched by two electron withdrawing groups and having high acidity are easily deprotonated by the stabilization effect of carbanion generated on the carbon to give anions. Therefore, carbanions can be easily generated in the presence of a base having basicity to such a degree as that of amine and alkoxide. The generated carbanions function as a nucleophile having active reactivity, and can be subjected to the Michael addition reaction with various electrophiles, for example, (meth)acrylates that are unsaturated carbonyl compounds.

The Michael addition reaction between an active methylene compound and an unsaturated carbonyl compound proceeds efficiently when pKa of active hydrogen of the active methylene compound is 15 or less. Preferred examples thereof include cyanoacetate (pKa=13.1) and acetoacetate (pKa=11.0). Cyanoacetate and acetoacetate are preferred in terms of the general versatility of raw materials.

As a hydrogen-abstraction catalyst that can be used in the Michael addition reaction, any appropriate catalyst can be used as long as the catalyst has a hydrogen-attraction effect. The catalyst includes, as amine-based catalysts, basic ionic liquids such as proline, triazabicyclodecene (TBD), diazabicyclo undecene (DBU), hexahydromethylpyrimidopyrimidine (MTBD), diazabicyclo nonane (DBN), tetramethyl guanidine (TMG), diazabicyclooctane (DABCO), catalysts in which TBD is carried on a solid-phase such as cross-linked polystyrene or silica gel, and butylmethylimidazolium hydroxide. Further, examples of the base catalyst may include sodium methoxide, sodium ethoxide, potassium tertiary butoxide, potassium hydroxide, sodium hydroxide, sodium metal, lithium diisopropyl amide (LDA), and butyl lithium. Further, organic metal catalysts include: ruthenium-based catalysts such as ruthenium cyclooctadiene cyclooctatriene and hydridoruthenium; iron-based catalysts such as trichloride iron and iron acetylacetonate; nickel-based catalysts such as nickel acetyl acetonate, nickel acetate, and nickel salicyl aldehyde; copper-based catalysts; palladium-based catalysts; scandium-based catalysts; lanthanum-based catalysts; and ytterbium-based catalysts. Of those, from the viewpoint of reactivity and the small degree of side reactions and staining, and the versatility of reagent, amine-based catalysts and base catalysts are preferable. The catalysts may be used alone or in combination.

The use amount of the hydrogen-abstraction catalyst may be a catalyst amount with respect to raw materials. When the use amount is too large, a side reaction may be caused, and when the use amount is too small, the reaction may not proceed. The use amount is preferably 0.0001 to 100 mol %, more preferably 0.01 to 10 mol %, and still more preferably 0.1 to 10 mol %.

The reaction temperature of the Michael addition reaction is preferably −78 to 200° C., more preferably 0 to 80° C., and still more preferably around room temperature, i.e., about 25° C. (15 to 35° C.).

The reaction time of the Michael addition reaction is preferably 10 seconds to 1 week, more preferably 1 minute to 10 hours, and still more preferably 3 minutes to 5 hours. The reaction may be completed appropriately by checking the reaction progress by analysis means such as thin film chromatography (TLC), high-performance liquid chromatography (HPCL), NMR, and infrared spectroscopy.

As the reaction solvent to be used in the Michael addition reaction, any suitable solvent can be adopted as long as it does not react with the hydrogen-abstraction catalyst to be used, does not react with or decompose a base, and preferably dissolves a raw material compound. For example, a solvent that dissolves an intended substance due to the final increase in solubility of a liquid crystal compound may be used even though a raw material compound is not completely dissolved therein. The solvent is preferably a dehydrated solvent, but the reaction can also proceed with a solvent that is not dehydrated.

Hereinafter, specific production methods of a typical liquid crystal compound among the liquid crystal compounds of the present invention are described.

The liquid crystal compound of the present invention can be produced by a method shown in a general formula (6), for example, in the case where the liquid crystal compound of the present invention is a liquid crystal compound having a chemical structure represented by the general formula (5d), in which: in the general formula (1), X represents —CN and R¹ and R² each have a chemical structure represented by the general formula (2); and in the general formula (2), J represents —H, A represents an ethylene group, Y represents —O—, and L has a chemical structure represented by the general formula (4c) (in Z represents —COO—). That is, the liquid crystal compound can be produced by: synthesizing a substance through cyanoacetic acid-esterification of pentaerythritol that is tetrafunctional alcohol as a raw material; and performing Michael addition reaction by using a liquid crystal acrylic monomer.

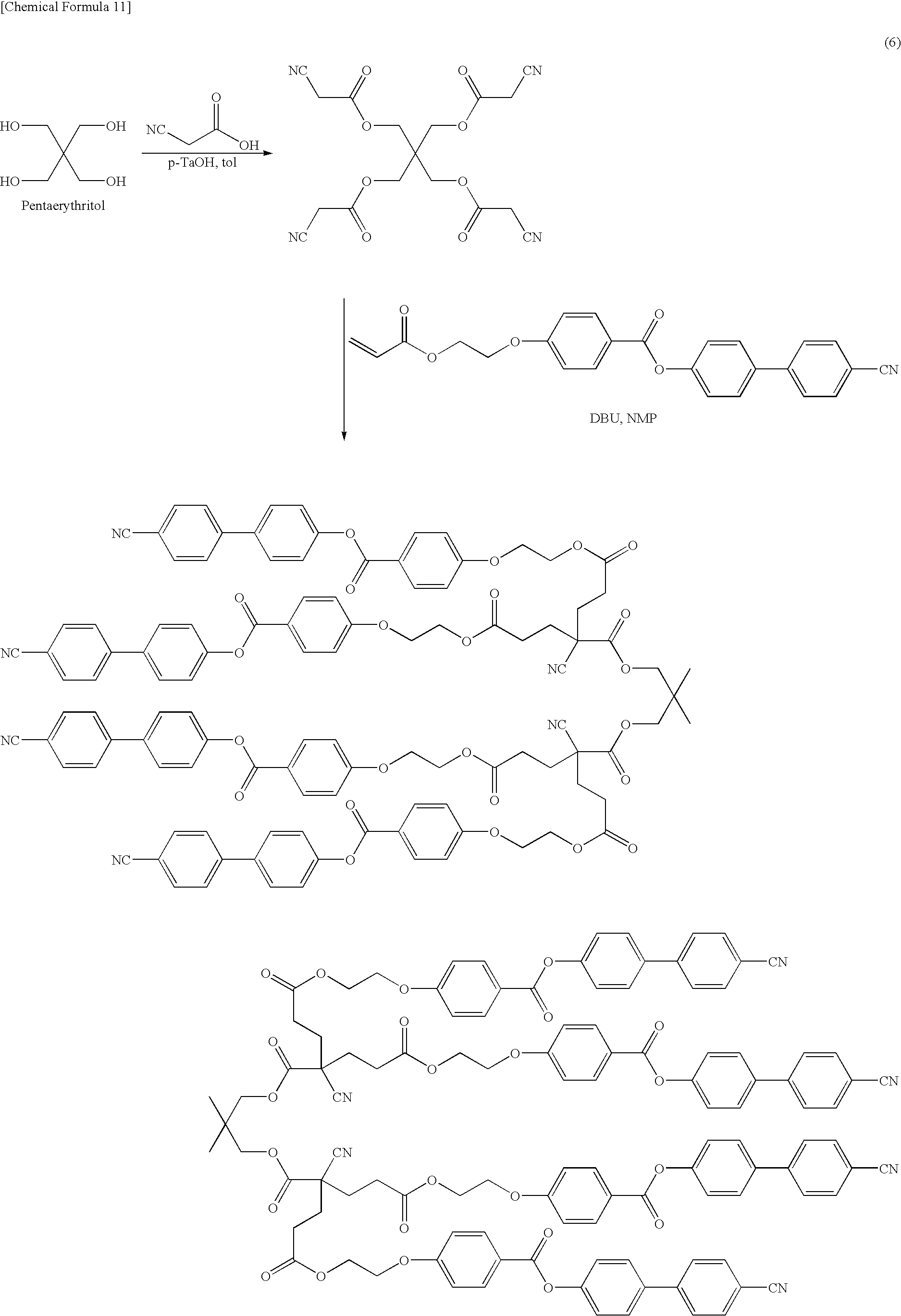

The liquid crystal compound of the present invention can be produced by a method shown in a general formula (7), for example, in the case where the liquid crystal compound of the present invention is an analogue of the liquid crystal compound produced by the method shown in the general formula (6) and has a chemical structure represented by the general formula (5b).

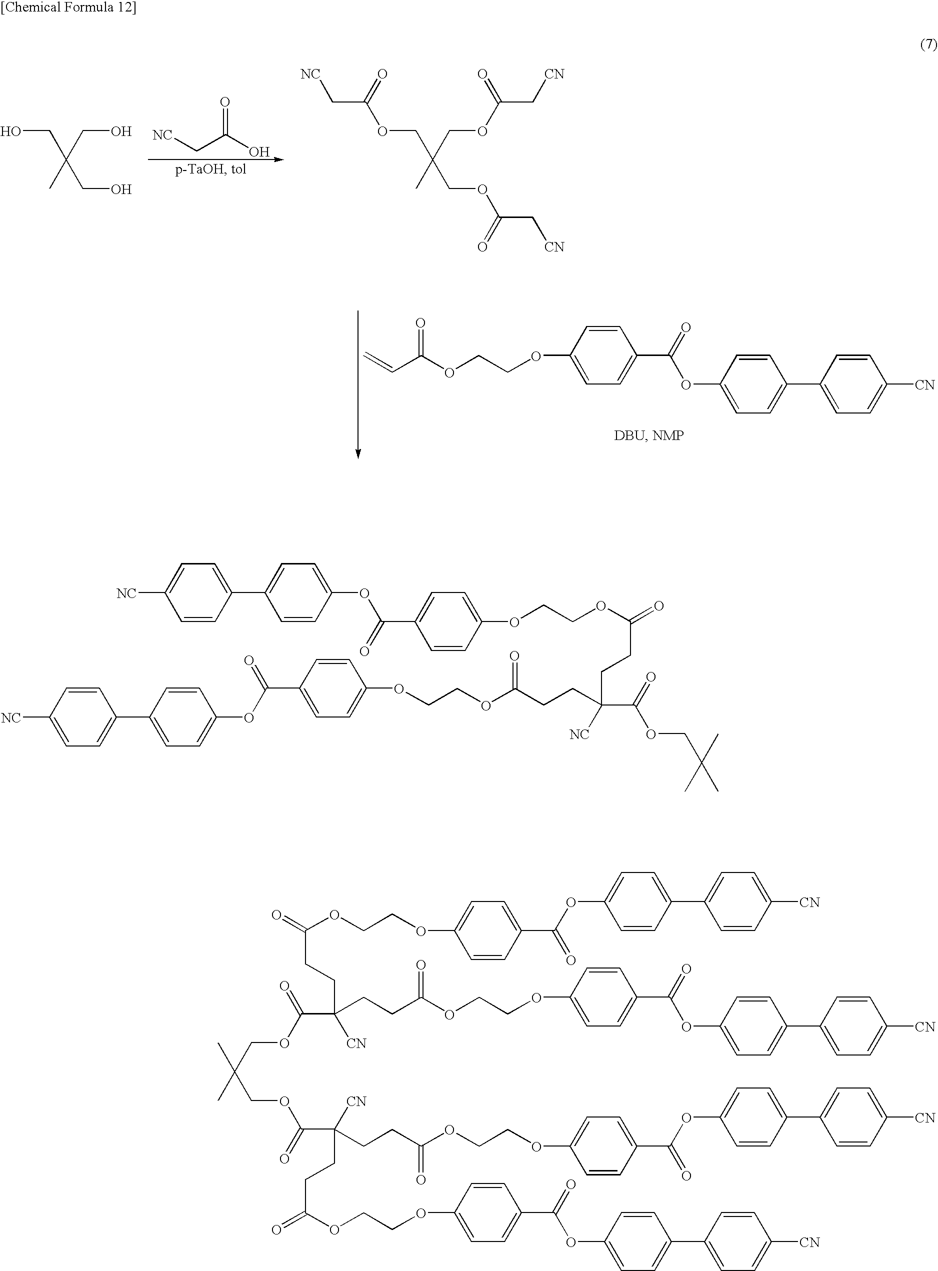

The liquid crystal compound of the present invention can be produced by a method shown in a general formula (8), for example, in the case where the liquid crystal compound of the present invention is an analogue of the liquid crystal compound produced by the method shown in the general formula (6) and has a chemical structure represented by the general formula (5e). That is, the liquid crystal compound can be produced by using dipentaerythritol as a raw material.

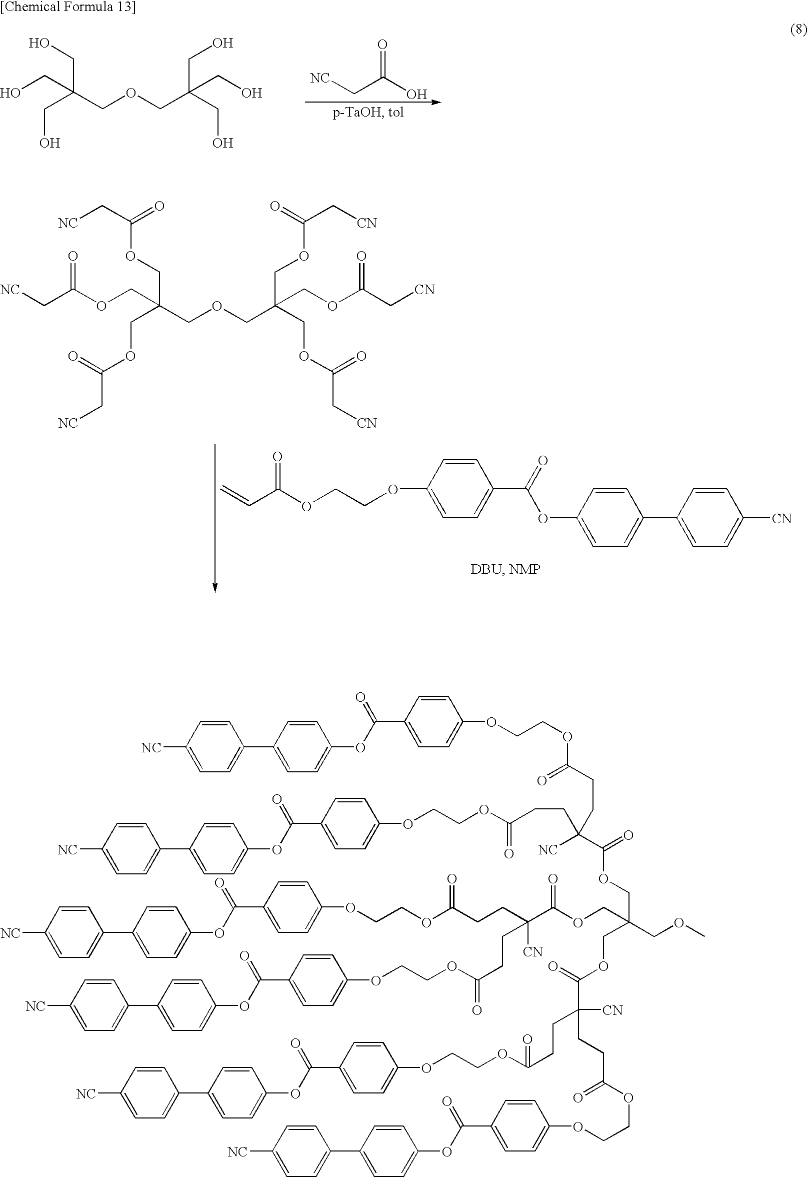

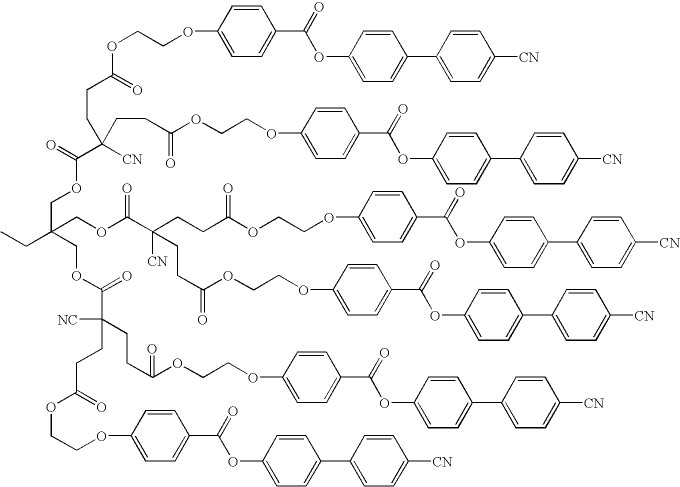

As shown in the general formulae (6) to (8), up to hexafunctional, octafunctional, and dodecafunctional liquid crystal site adducts can be synthesized, respectively, by using polyfunctional alcohols of trifunctional, tetrafunctional, and hexafunctional and liquid crystal acrylic monomers as raw materials. The polyfunctional alcohols as raw materials are not limited to difunctional, trifunctional, tetrafunctional, hexafunctional, and octafunctional alcohols each having a chemical structure represented by the general formulae (5a) to (5f) and can be used without limitation. From the viewpoint of reactivity, primary alcohol is preferably used.

A production example of a tetrafunctional liquid crystal acrylic adduct using, as a raw material, bis(hydroxymethyl)propionic acid as a polyfunctional alcohol is shown in a general formula (9). If there is produced a compound in which a carboxylic acid site of bis(hydroxymethyl)propionic acid and another site thereof (represented by R in the general formula (9)) are connected with each other by an ester bond, and further, cyanoacetic acid is separately subjected to esterification at a hydroxy (alcohol) site, the Michael addition reaction is finally performed by using the liquid crystal acrylic monomer, whereby a liquid crystal compound having four liquid crystal sites and R groups can be produced.

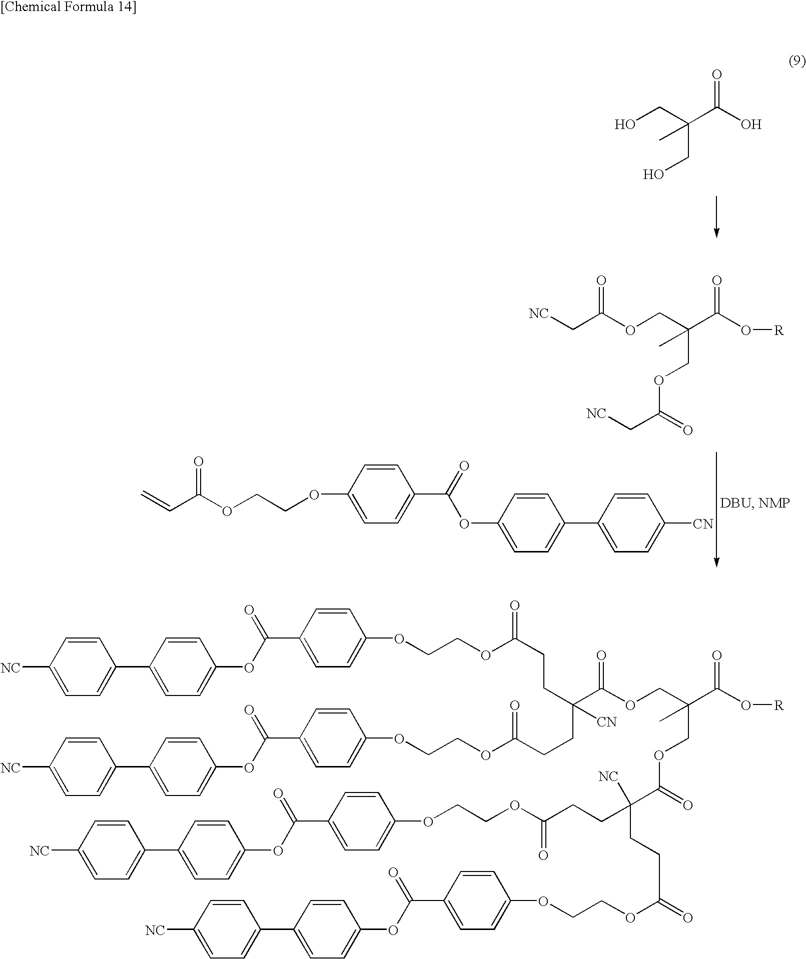

The liquid crystal compound of the present invention may be a cross-linkable liquid crystal compound. That is, the liquid crystal compound of the present invention may be a liquid crystal compound having a cross-linkable group. As the cross-linkable group, any appropriate group may be employed as long as the group is capable of performing a cross-linking reaction.

The liquid crystal compound of the present invention may be used alone or in combination.

The liquid crystal compound of the present invention has excellent compatibility. Thus, in order to express multiple functions, there is no need to introduce a plurality of functional sites into one liquid crystal compound, and target multiple functions can be expressed by blending a plurality of liquid crystal compounds and compatibilize the blend. Further, the liquid crystal compound of the present invention enables to give a film not having phase separation, because the liquid crystal compound has such an excellent compatibility.

The liquid crystal compound of the present invention can be used for various purposes in combination with other components. As the other components, any appropriate components in accordance with the purpose may be employed.

As the other components, any additives may be selected appropriately as long as the effect of the present invention is adversely affected. Specific examples thereof include an antioxidant, a flame retardant, a leveling agent, and a plasticizer. They may be used alone or in combination. Examples of the antioxidant include a phenol-based compound, an amine-based compound, an organic sulfur-based compound, and a phosphine-based compound.

The liquid crystal compound of the present invention may be adopted for any appropriate use. For example, the liquid crystal compound may be used for an optical element such as a retardation plate, a viewing angle compensation plate, or a cholesteric selective reflection plate, which utilizes a birefringent behavior of the liquid crystal compound of the present invention. Further, the liquid crystal compound can also be used to be developed into an optical recording material by combining a photoisomerization behavior therewith. Further, the liquid crystal compound of the present invention can be formed into a film, and can be changed into any appropriate form by any appropriate means such as solution application, e.g., spin coating, and heat-melting and then used.

### <<Optical Element>>

The optical element of the present invention includes the liquid crystal compound of the present invention. Further, the liquid crystal compound of the present invention includes a cross-linked product formed by cross-linking the liquid crystal compound of the present invention which includes the cross-linkable liquid crystal compound.

As the kind of the optical element of the present invention, any appropriate kind thereof may be employed. Examples thereof include a retardation plate, a viewing angle compensation plate, and a cholesteric selective reflection plate.

### <<Polarizing Plate>>

The polarizing plate of the present invention includes the optical element of the present invention. The polarizing plate of the present invention preferably includes a polarizer formed of a polyvinyl alcohol-based resin, a polarizer protective film formed on at least one side of the polarizer, and the optical element of the present invention. The polarizer is preferably formed by being bonded to the polarizer protective film via an adhesive layer.

In a preferred embodiment of the polarizing plate of the present invention, the polarizing plate is formed by laminating at least one optical element on at least one surface of a laminate of polarizer protective film/polarizer/polarizer protective film. The polarizer protective films laminated on both surfaces of the polarizer may be the optical elements of the present invention.

As a polarizer, any appropriate polarizer can be adopted or used as long as the polarizer is a film capable of converting natural light or polarized light into any polarized light. For example, the polarizer preferably used converts natural light or polarized light into linearly polarized light. When incident light is split into two perpendicular polarization components, used is a polarizer that has a function of transmitting one of the polarization components and has at least one function selected from the functions of absorbing, reflecting, and scattering the other polarization component.

As a thickness of the polarizer, any appropriate thickness may be employed. The thickness of the polarizer is preferably 5 μm to 80 μm. When the thickness is in the above range, the polarizer having excellent optical properties and mechanical strength can be obtained.

As the polarizer protective film, any appropriate film which can be used as a protective film for a polarizer may be employed. Specific examples of a material used as a main component of the film include a cellulose-based resin such as triacetylcellulose (TAC), and transparent resins such as a polyester-based resin, a polyvinyl alcohol-based resin, a polycarbonate-based resin, a polyamide-based resin, a polyimide-based resin, a polyether sulfone-based resin, a polysulfone-based resin, a polystyrene-based resin, a polynorbornene-based resin, a polyolefin-based resin, an acrylic resin, and an acetate-based resin. Another example thereof includes an acrylic, urethane-based, acrylic urethane-based, epoxy-based, or silicone-based thermosetting resin and UV-curing resin. Still another example thereof includes a glassy polymer such as a siloxane-based polymer. Further, a polymer film described in JP 2001-343529 A (WO 01/37007) may also be used. As a material for the film, used is a resin composition containing a thermoplastic resin having a substituted or unsubstituted imide group on a side chain, and a thermoplastic resin having a substituted or unsubstituted phenyl group and a nitrile group on a side chain. A specific example thereof includes a resin composition containing an alternate copolymer of isobutene and N-methylmaleimide and an acrylonitrile/styrene copolymer. The polymer film may be an extruded product of the above-mentioned resin composition, for example. Of those, TAC, a polyimide-based resin, a polyvinyl alcohol-based resin, and a glassy polymer are preferable. Each of the polarizer protective film may be the same or different from each other.

The polarizer protective film is preferably transparent and colorless. To be specific, the polarizer protective film has a thickness direction retardation value of preferably −90 nm to +90 nm, more preferably −80 nm to +80 nm, and most preferably −70 nm to +70 nm.

The polarizer protective film may have any appropriate thickness as long as the preferable thickness direction retardation can be obtained. To be specific, the thickness of the polarizer protective film is preferably 5 mm or less, more preferably 1 mm or less, particularly preferably 1 to 500 μm, and most preferably 5 to 150 μm.

In the present invention, each layer such as the polarizer and the optical element forming the polarizing plate may be provided with UV absorbability through a method for treatment with a UV absorber such as a salicylate-based compound, a benzophenol-based compound, a benzotriazole-based compound, a cyanoacrylate-based compound, or a nickel complex salt-based compound or the like.

The polarizing plate of the present invention may be provided on one of a viewer side and a backlight side of a liquid crystal cell, or on both sides thereof, and is not limited.

### <<Image Display Apparatus>>

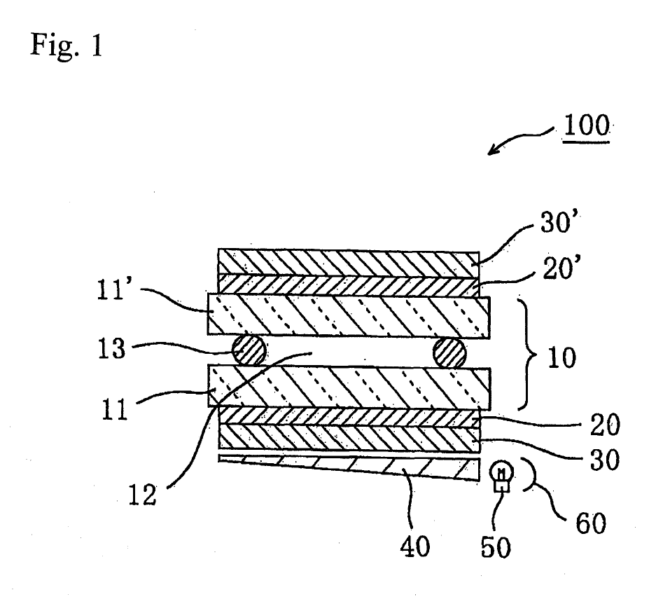

The image display apparatus of the present invention is described. The image display apparatus of the present invention includes at least one polarizing plate of the present invention. Here, a description is made on a liquid crystal display apparatus as an example, but it goes without saying that the present invention can be adopted for all the display apparatuses which require the polarizing plates. Specific examples of the image display apparatus which the polarizing plate of the present invention can be adopted for include self-luminous display apparatuses such as an electroluminescence (EL) display, a plasma display (PD), and a field emission display (FED). FIG. 1 is a schematic cross-sectional view of a liquid crystal display apparatus according to a preferred embodiment of the present invention. A transmission-type liquid crystal display apparatus is described in the example shown in the diagram, but it goes without saying that the present invention can be adopted for a reflection-type liquid crystal display apparatus and the like.

A liquid crystal display apparatus 100 includes a liquid crystal cell 10, retardation films 20, 20′ placed in a manner of sandwiching the liquid crystal cell 10, polarizing plates 30, 30′ placed outer sides of the retardation films 20, 20′, a light guide plate 40, a light source 50, and a reflector 60. The polarizing plates 30, 30′ are placed in such a manner that polarizing axes thereof are perpendicular to each other. The liquid crystal cell 10 includes a pair of glass substrates 11, 11′ and a liquid crystal layer 12 as a display medium interposed between the substrates 11, 11′. On one substrate 11, switching elements (typically TFTs) controlling the electrooptical properties of liquid crystal, scanning lines supplying a gate signal to the switching elements, and signal lines supplying a source signal are provided (neither of them is shown). On the other glass substrate 11′, a color layer forming a color filter and a light-shielding layer (a black matrix layer) are provided (neither of them is shown). The interval (cell gap) between the substrates 11, 11′ is controlled with a spacer 13. In the liquid crystal display apparatus of the present invention, the above-mentioned polarizing plate of the present invention is employed as at least one of the polarizing plates 30, 30′.

For example, in the case of a TN system, in the liquid crystal display apparatus 100, liquid crystal molecules of the liquid crystal layer 12 are aligned in such a manner that polarization axes are shifted by 90° under no voltage application. In such a state, incident light with a light in one direction transmitted by the polarizing plate is twisted by 90° by liquid crystal molecules. As described above, since the polarizing plates are placed so that polarization axes thereof are perpendicular to each other, the light (polarized light) having reached the other polarizing plate is transmitted through the polarizing plate. Thus, under no voltage application, the liquid crystal display apparatus 100 exhibits a white display (normally white system). On the other hand, when a voltage is applied to the liquid crystal display apparatus 100, the alignment of the liquid crystal molecules in the liquid crystal layer 12 changes. Consequently, light (polarized light) having reached the other polarizing plate cannot be transmitted through the polarizing plate and exhibits a black display. By switching a display in such a manner for each pixel using an active element, an image is formed.

### <<Optical Recording Material>>

The optical recording material of the present invention contains the liquid crystal compound of the present invention. The optical recording material of the present invention can be produced by applying a liquid crystal composition containing the liquid crystal compound of the present invention onto a substrate having an alignment regulating force, and subjecting the liquid crystal composition to heating alignment treatment, followed by cooling to room temperature. Further, the optical recording material of the present invention can also be produced by placing the liquid crystal composition containing the liquid crystal compound of the present invention between two substrates at least one of which has an alignment regulating force, and subjecting the liquid crystal composition to heating alignment treatment, followed by cooling to room temperature.

As the substrate (alignment substrate) having an alignment regulating force, there is no particular limit as long as the substrate can align a liquid crystal composition containing a polyfunctional compound of the present invention. For example, a plastic film or sheet whose surface is subjected to rubbing treatment with rayon cloth or the like can be used.

Examples of the plastic are not particularly limited and may include triacetyl cellulose (TAC), polyolefin such as polyethylene, polypropylene, or poly(4-methylpentene-1), polyimide, polyimideamide, polyetherimide, polyamide, polyetherether ketone, polyether ketone, polyketone sulfide, polyether sulfone, polysulfone, polyphenylene sulfide, polyphenylene oxide, polyethylene terephthalate, polybutylene terephthalate, polyethylene naphthalate, polyacetal, polycarbonate, polyarylate, acrylic resin, polyvinyl alcohol, polytetrafluoro ethylene, polynorbornene, cellulose-based plastics, an epoxy resin, and a phenol resin. A substrate made of metal such as aluminum, copper, or iron, a ceramic substrate, a glass substrate, or the like, on which the above-mentioned plastic film or sheet is placed, which is subjected to ITO treatment, on which a SiO₂ oblique deposition film is formed, or the like, can be used. Further, a laminate in which a stretched film having birefringence and being subjected to stretching treatment such as uniaxial stretching or the like is laminated as an alignment film on the above-mentioned plastic film or sheet can be used as an alignment substrate. Further, it is preferred that the substrate itself have birefringence, because the rubbing treatment, the lamination of a birefringence film on the surface, and the like are not necessary. As a method of providing a substrate itself with birefringence, there is a method of performing casting, extrusion molding, or the like, for example, in addition to the stretching treatment in formation of a substrate. In the case where a substrate subjected to alignment treatment is not used, there is also a method of producing an alignment substrate using an electric field or a magnetic field.

In the case where alignment regulation is not required, the liquid crystal composition can be formed on the substrate having no alignment regulating force.

As a method of applying the liquid crystal composition containing a polyfunctional compound of the present invention onto a substrate having an alignment regulating force, the liquid crystal composition may be allowed to flow by, for example, roll coating, spin coating, wire bar coating, dip coating, extrusion coating, curtain coating, or spray coating. Of those, spin coating and extrusion coating are preferred in terms of application efficiency.

The temperature conditions of heating alignment treatment after the above application can be appropriately determined in accordance with, for example, the kind of a liquid crystal compound to be used, specifically, a temperature at which the liquid crystal compound exhibits liquid crystallinity. Further, the liquid crystal composition is cooled to room temperature after being subjected to heating alignment treatment, whereby the liquid crystal composition is vitrified and can express an anisotropy function.

### EXAMPLES

Hereinafter, the present invention is described specifically by way of examples, but the present invention is not limited thereto. Unless otherwise specified, the part and percent in examples are expressed in terms of weight.

### Example 1

To a toluene suspension (200 mL) of pentaerythritol (5 g, 37 mmol) and cyanoacetic acid (18.7 g, 220 mmol), a catalytic amount of p-toluenesulfonic acid monohydrate was added, and a Dean-Stark tube was set. The mixture was heat-stirred at 140° C. for 3 hours, and water generated by the reaction was removed together with toluene. The reaction solution was returned to room temperature, and a saturated sodium hydrogen carbonate aqueous solution was added, whereby the deposited precipitate was filtered. The deposited precipitate was washed with a saturated sodium hydrogen carbonate aqueous solution and water, and then was heat-dried under reduced pressure, whereby a tetrafunctional cyanoacetate compound (13.4 g, 33 mmol, 90%) was obtained.

The tetrafunctional cyanoacetate compound (0.5 g, 1.24 mmol) and acrylate having a liquid crystal group (4.09 g, 9.89 mmol) were dissolved in 50 mL of dimethylformamide (DMF) under a nitrogen atmosphere, 5 drops of diazabicycloundecene (DBU) were added thereto, and the mixture was stirred at 50° C. for 3 hours. 10 drops of 3 mol/L hydrochloric acid were added to the reaction solution to thereby neutralize the reaction solution, and then the resultant was reprecipitated in methanol, whereby the generated precipitate was filtered. Again, the resultant was dissolved in tetrahydrofuran and reprecipitated in methanol, whereby the generated precipitate was filtered. The resultant was then heated under vacuum, whereby a liquid crystal compound (1) (4.33 g, 1.16 mmol, 94%) was obtained.

The obtained liquid crystal compound (1) (molecular weight: 3711.7) was measured for a mass spectrometry by MALDI-TOF MS measurement, and as a result, ions having m/z of 3744.6 were mainly detected. Those ions corresponded to ions in which sodium ions were added to the liquid crystal compounds (1), whereby it was found that the liquid crystal compound (1) was obtained.

A synthesis scheme of the liquid crystal compound (1) is shown in a general formula (10).

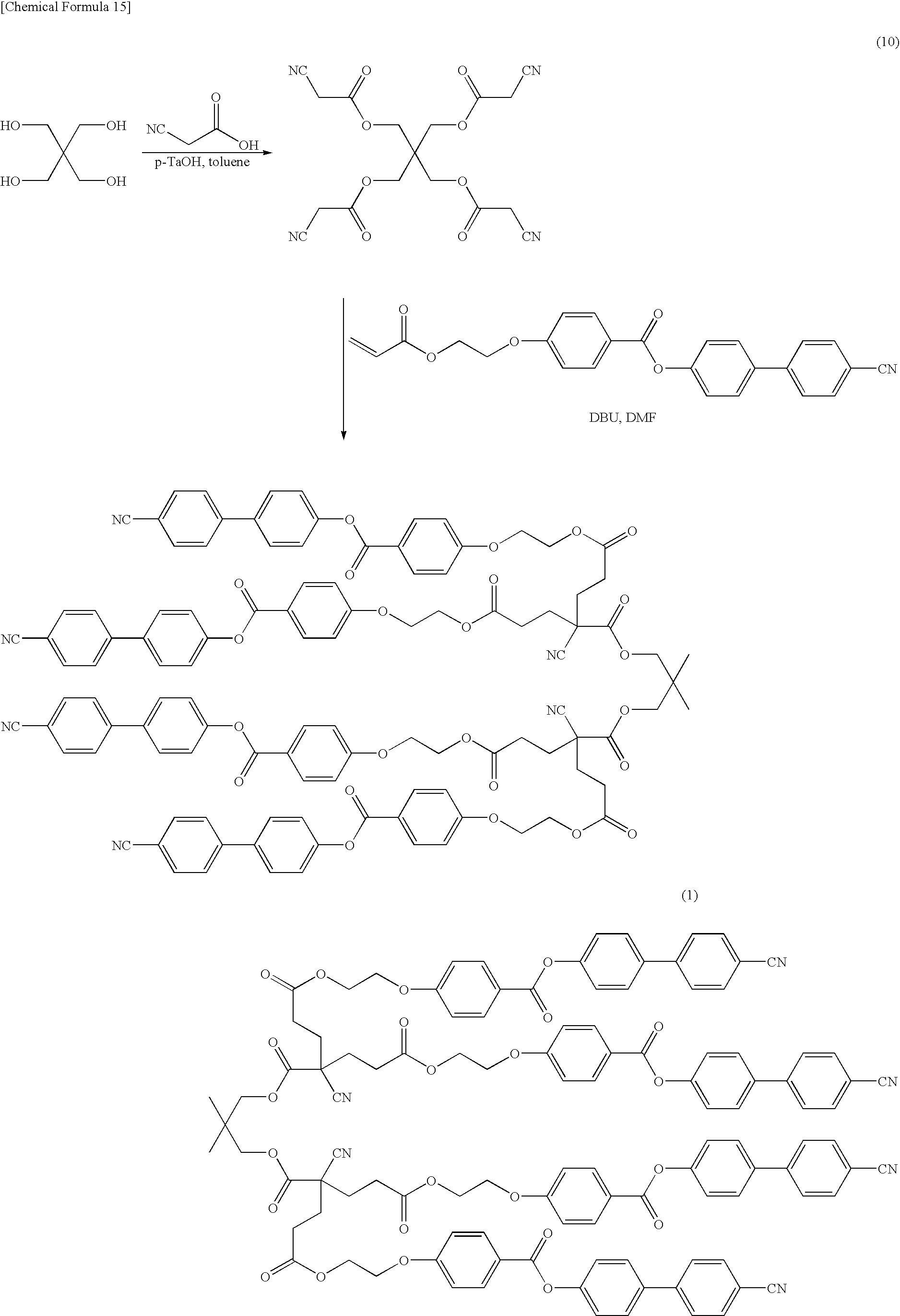

### Example 2

To a toluene suspension (600 mL) of tris(hydroxymethyl)ethane (15 g, 124 mmol) and cyanoacetic acid (42.5 g, 499 mmol), p-toluenesulfonic acid monohydrate (3g) was added, and a Dean-Stark tube was set. The mixture was heat-stirred at 140° C. for 3 hours, and water generated by the reaction was removed together with toluene. The reaction solution was returned to room temperature, and a saturated sodium hydrogen carbonate aqueous solution was added, whereby the deposited precipitate was filtered. The deposited precipitate was washed with a saturated sodium hydrogen carbonate aqueous solution and water, and then was heat-dried under reduced pressure, whereby a trifunctional cyanoacetate compound (32.8 g, 102 mmol, 82%) was obtained.

The trifunctional cyanoacetate compound (0.637 g, 1.98=1) and acrylate having a liquid crystal group (5 g, 12.1 mmol) were dissolved in 50 mL of dimethylformamide (DMF) under a nitrogen atmosphere, 5 drops of diazabicycloundecene (DBU) were added thereto, and the mixture was stirred at 50° C. for 3 hours. 10 drops of 3 mol/L hydrochloric acid were added to the reaction solution to thereby neutralize the reaction solution, and then the resultant was reprecipitated in methanol, whereby the generated precipitate was filtered. Again, the resultant was dissolved in tetrahydrofuran and reprecipitated in methanol, whereby the generated precipitate was filtered. The resultant was then heated under vacuum, whereby a liquid crystal compound (2) (5.22 g, 1.86 mmol, 94%) was obtained.

The obtained liquid crystal compound (2) (molecular weight: 2801.8) was measured for mass spectrometry by MALDI-TOF MS measurement, and as a result, ions having m/z of 2832.5 were mainly detected. Those ions corresponded to ions in which sodium ions were added to the liquid crystal compounds (2), whereby it was found that the liquid crystal compound (2) was obtained.

A synthesis scheme of the liquid crystal compound (2) is shown in a general formula (11) .

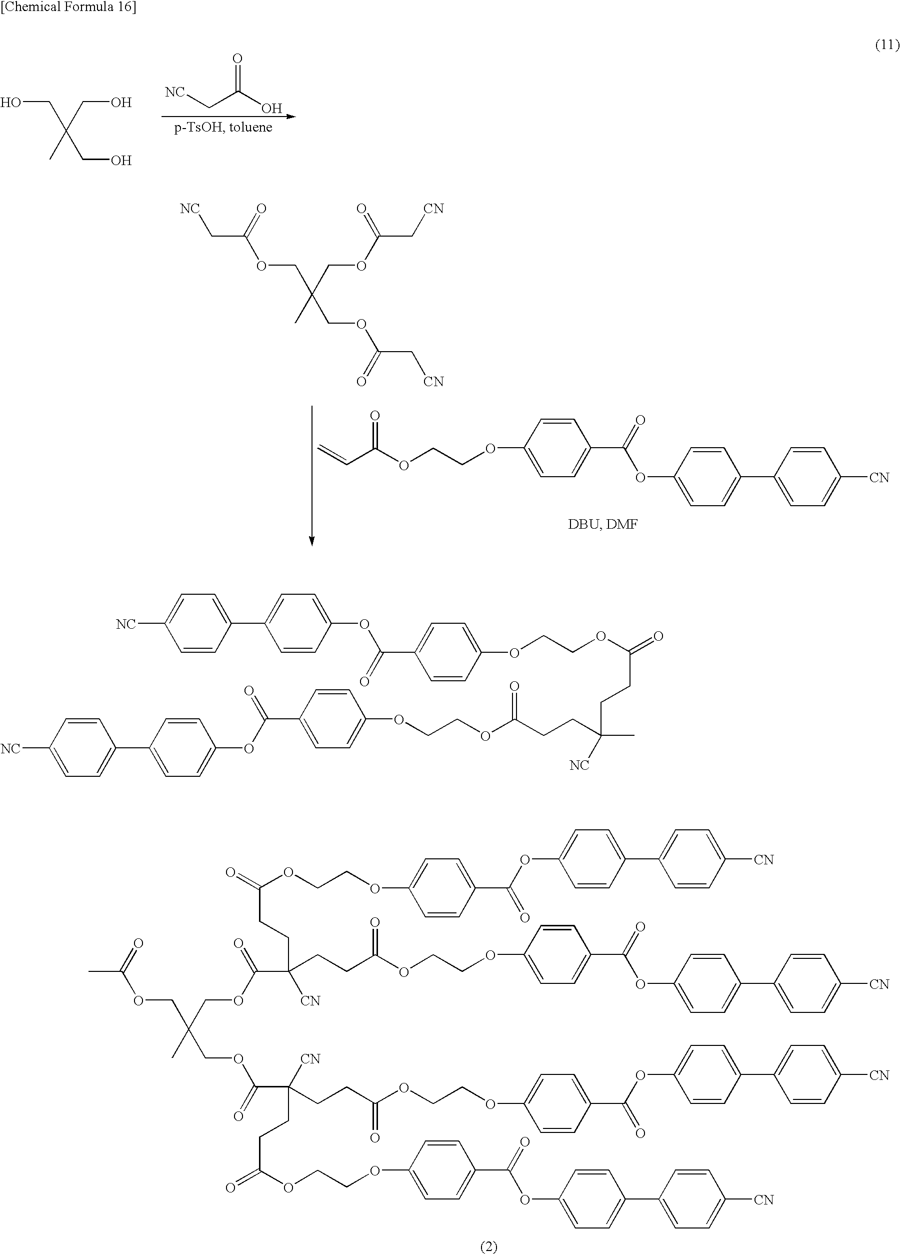

### Example 3

To a toluene suspension (400 mL) of dipentaerythritol (5 g, 19.7 mmol) and cyanoacetic acid (13.38 g, 157 mmol), p-toluenesulfonic acid monohydrate (2g) was added, and a Dean-Stark tube was set. The mixture was heat-stirred at 140° C. for 3 hours, and water generated by the reaction was removed together with toluene. The reaction solution was returned to room temperature, and a saturated sodium hydrogen carbonate aqueous solution was added, whereby the deposited precipitate was filtered. The deposited precipitate was washed with a saturated sodium hydrogen carbonate aqueous solution, water, and methanol, and then was heat-dried under reduced pressure, whereby a hexafunctional cyanoacetate compound (11.7 g, 17.9 mmol, 91%) was obtained.

The hexafunctional cyanoacetate compound (0.5 g, 0.76 mmol) and acrylate having a liquid crystal group (3.77 g, 9.13 mmol) were dissolved in 30 mL of dimethylformamide (DMF) under a nitrogen atmosphere, 5 drops of diazabicycloundecene (DBU) were added thereto, and the mixture was stirred at 50° C. for 3 hours. 10 drops of 3 mol/L hydrochloric acid were added to the reaction solution to thereby neutralize the reaction solution, and then the resultant was reprecipitated in methanol, whereby the generated precipitate was filtered. Again, the resultant was dissolved in tetrahydrofuran and reprecipitated in methanol, whereby the generated precipitate was filtered. The resultant was then heated under vacuum, whereby a liquid crystal compound (3) was obtained.

The obtained liquid crystal compound (3) (molecular weight: 5617.6) was measured for mass spectrometry by MALDI-TOF MS measurement, and as a result, ions having m/z of 5650.5 were mainly detected. Those ions corresponded to ions in which sodium ions were added to the liquid crystal compounds (3), whereby it was found that the liquid crystal compound (3) was obtained.

A synthesis scheme of the liquid crystal compound (3) is shown in a general formula (12) .

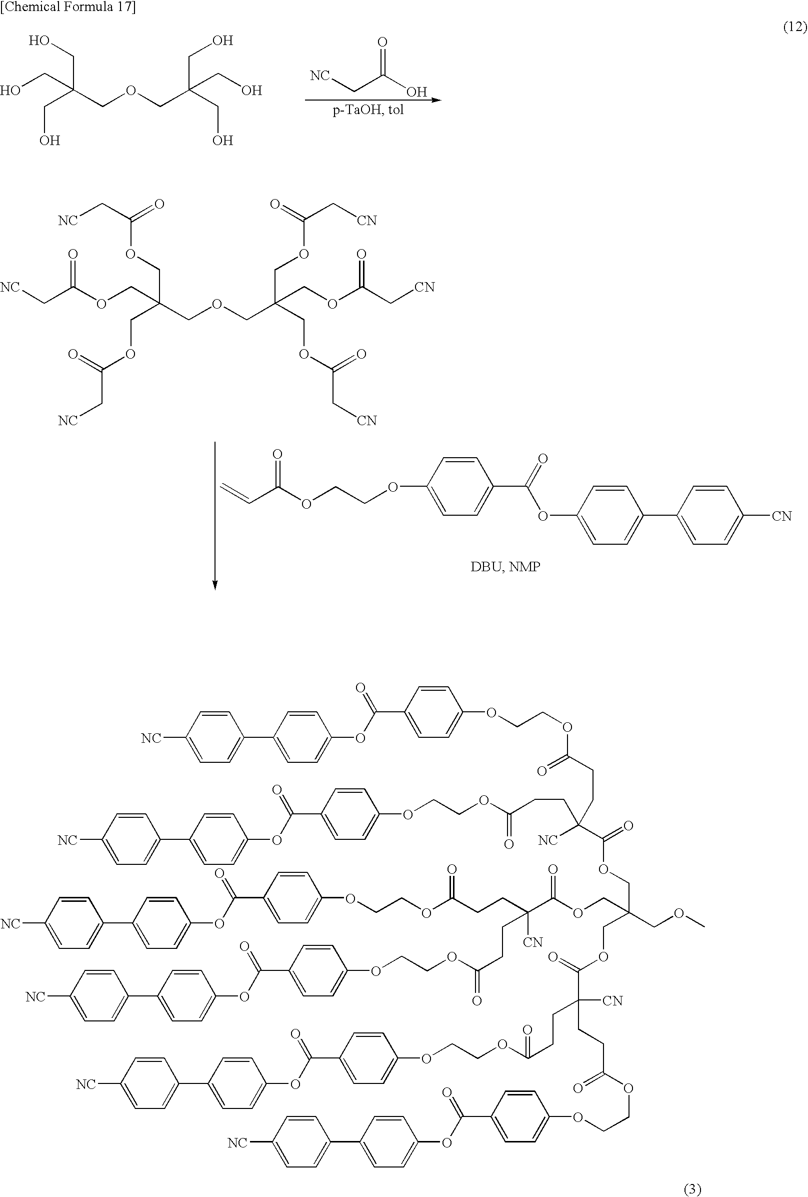

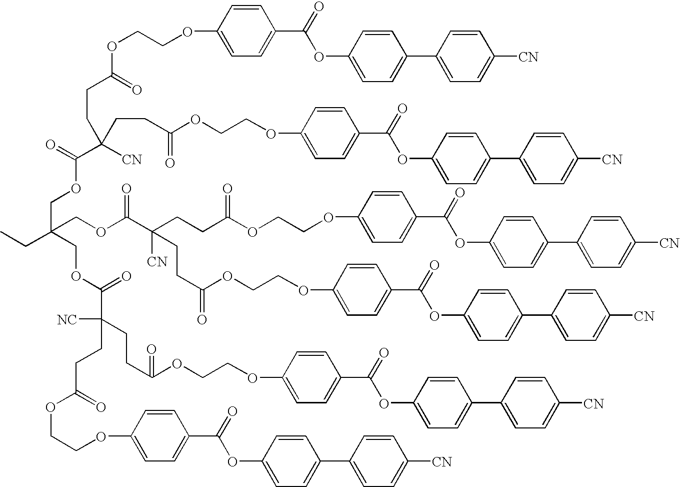

### Example 4

The tetrafunctional cyanoacetate compound obtained in Example 1 (0.5 g, 1.24 mmol) and acrylate having a liquid crystal group (3.07 g, 7.42 mmol) were dissolved in 50 mL of dimethylformamide (DMF) under a nitrogen atmosphere, 5 drops of diazabicycloundecene (DBU) were added thereto, and the mixture was stirred at 50° C. for 3 hours. Next, 1,6-hexanedioldiacrylate (1.1 mL, 4.95 mmol) was added thereto, and the mixture was stirred at 50° C. for 1 hour. 10 drops of 3 mol/L hydrochloric acid were added to the reaction solution to thereby neutralize the reaction solution, and then the resultant was reprecipitated in methanol, whereby the generated precipitate was filtered. Again, the resultant was dissolved in tetrahydrofuran and reprecipitated in methanol, whereby the generated precipitate was filtered. The resultant was then heated under vacuum, whereby a liquid crystal compound (4) was obtained (3.3 g).

The obtained liquid crystal compound (4) was measured for mass spectrometry by MALDI-TOF MS measurement. In Example 1, obtained was the liquid crystal compound in which liquid crystal groups (LC) were added to 8 sites of the tetrafunctional cyanoacetate core, but according to Example 4, it was found that, in addition to the adduct having 8 sites of the liquid crystal group (LC), an adduct in which 1 site of hexanedioldiacrylate (Ac) was added to each of 7 sites of the liquid crystal group, an adduct of 6 sites of LC+2 sites of Ac, an adduct of 5 sites of LC+3 sites of AC, and an adduct of 4 sites of LC+4 sites of Ac were obtained.

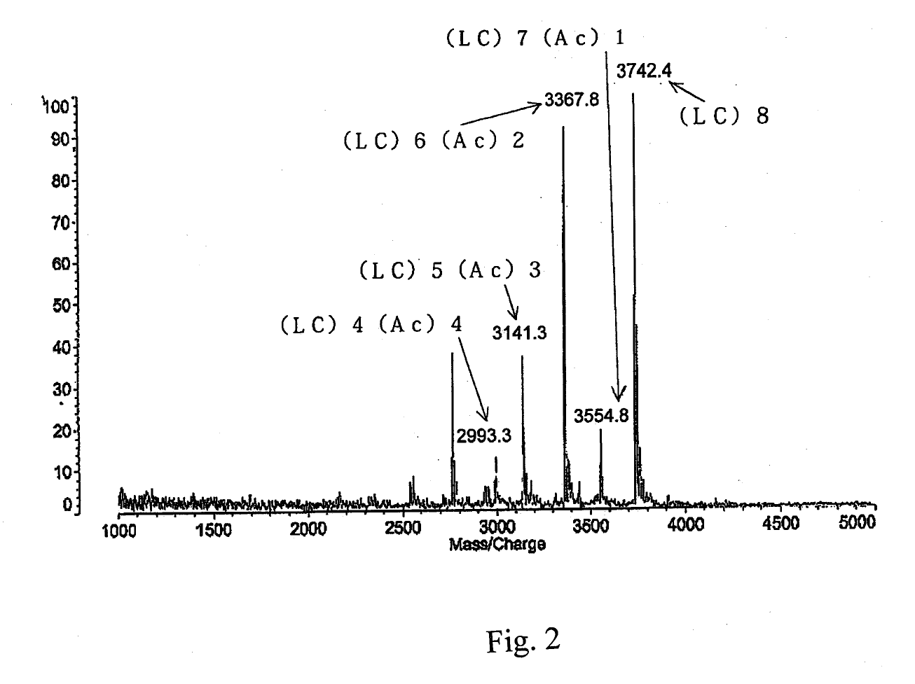

A synthesis scheme of the liquid crystal compound (4) is shown in a general formula (13). Further, a mass spectrum of the liquid crystal compound (4) is shown in FIG. 2.

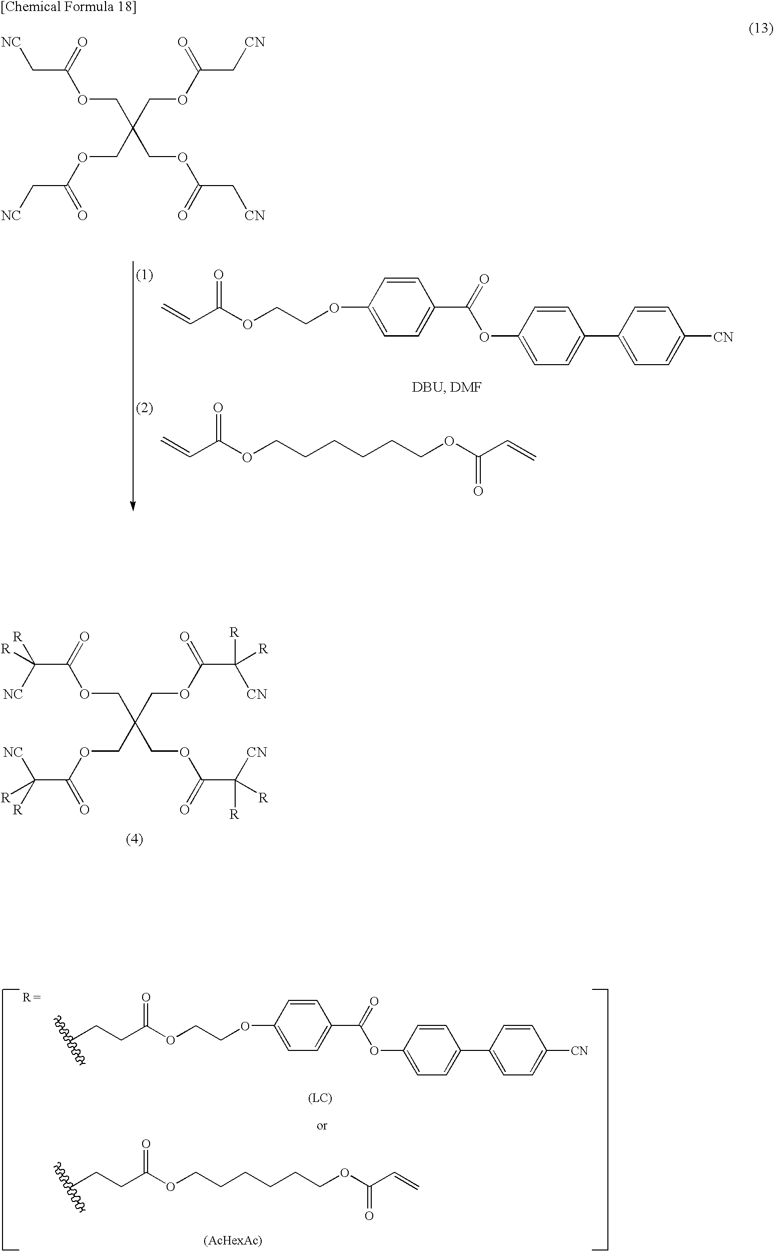

### Example 5

The tetrafunctional cyanoacetate compound obtained in Example 1 (0.5 g, 1.24 mmol) and acrylate having a liquid crystal group (3.59 g, 8.68 mmol) were dissolved in 50 mL of dimethylformamide (DMF) under a nitrogen atmosphere, 5 drops of diazabicycloundecene (DBU) were added thereto, and the mixture was stirred at 50° C. for 3 hours. Next, ethylene glycol acrylate methacrylate (0.79 g, 3.71 mmol) was added thereto, and the mixture was stirred at 50° C. for 1 hour. 10 drops of 3 mol/L hydrochloric acid were added to the reaction solution to thereby neutralize the reaction solution, and then the resultant was reprecipitated in methanol, whereby the generated precipitate was filtered. Again, the resultant was dissolved in tetrahydrofuran and reprecipitated in methanol, whereby the generated precipitate was filtered. The resultant was then heated under vacuum, whereby a liquid crystal compound (5) was obtained (3.2 g).

The obtained liquid crystal compound (5) was measured for mass spectrometry by MALDI-TOF MS measurement. In Example 1, obtained was the liquid crystal compound in which liquid crystal groups (LC) were added to 8 sites of the tetrafunctional cyanoacetate core, but according to Example 5, it was found that, in addition to the adduct having 8 sites of the liquid crystal group (LC), an adduct in which 1 site of ethylene glycol acrylate methacrylate was added to each of 7 sites of the liquid crystal group was obtained.

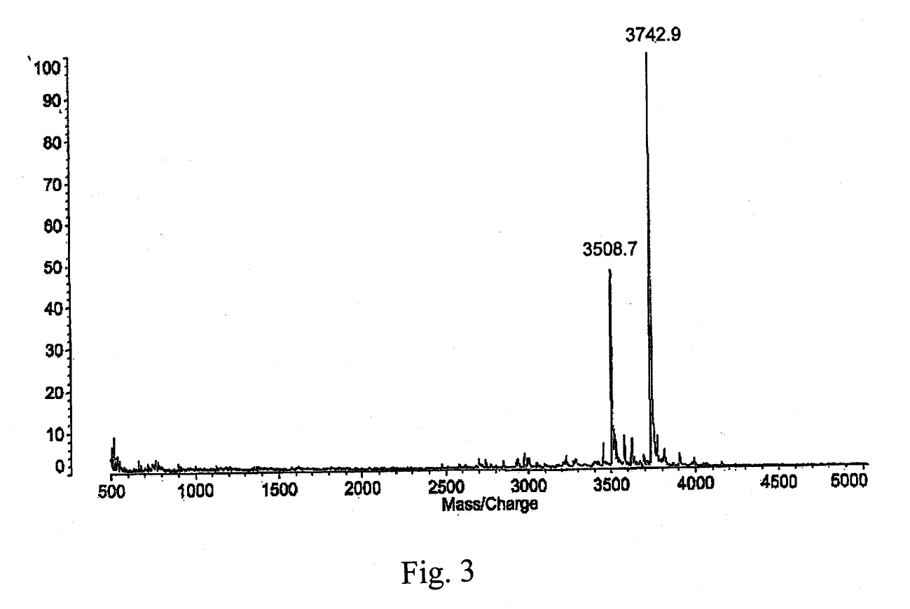

A mass spectrum of the liquid crystal compound (5) is shown in FIG. 3.

### Example 6

An aligned film was fabricated by using the liquid crystal compounds (1) to (5) obtained in Examples 1 to 5. Each of 25 wt % of cyclohexanone solutions of the liquid crystal compounds (1) to (5) was applied by spin coating onto a glass plate on which a polyvinyl alcohol alignment layer had been formed. Then, in order to volatilize the solvent and to perform liquid crystal alignment, the resultant was heat-treated at 180° C. for 120 seconds, whereby a uniaxially aligned optical element in which the liquid crystal compound formed a nematic alignment state was produced. Subsequently, the uniaxially aligned optical element was left standing to cool at room temperature, whereby the element was fixed to the glass and the uniaxial alignment state thereof was maintained.

### INDUSTRIAL APPLICABILITY

The liquid crystal compound of the present invention can be used for an optical element, a polarizing plate, an image display apparatus, and an optical recording material.

1. A liquid crystal compound, comprising two or more chemical structures Q each represented by a general formula (1): 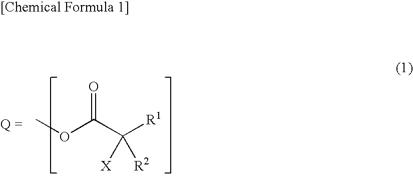
where, in the general formula (1): X represents one of —CN and —COCH₃; and R¹ and R² each independently represent any one of —H, a chemical structure represented by a general formula (2), and a chemical structure represented by any one of general formulae (3a) to (3f): 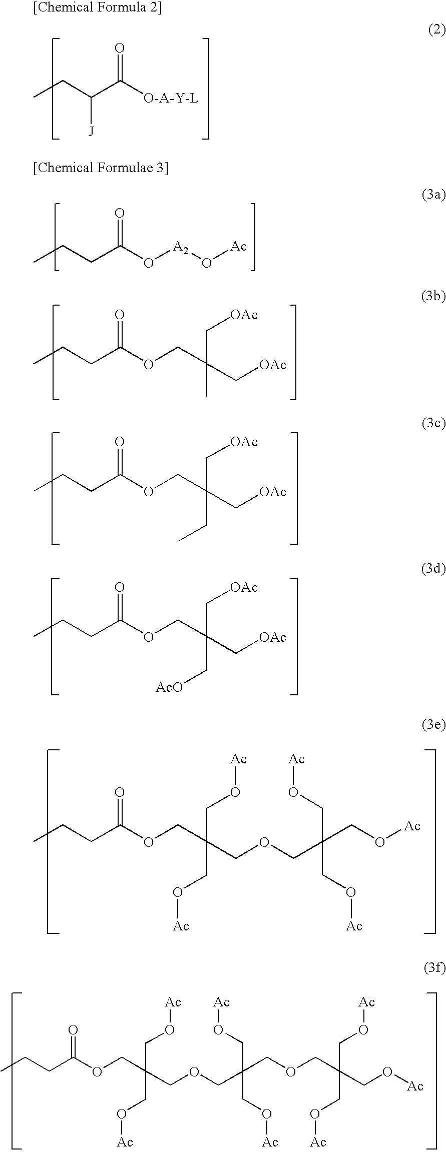
where, in the general formula (2): J represents one of —H and —CH₃; A represents a single bond or an alkylene group having 2 to 12 carbon atoms, in which one —CH₂— or two or more nonadjacent —CH₂—'s present in the alkylene group may be substituted with —O—; Y represents any one of —O—, —COO—, —OCO—, and —OCOO—; and L represents a chemical structure represented by any one of general formulae (4a) to (4g),
where, in the general formulae (3a) to (3f): Ac represents a (meth)acryloyl group; and A₂ represents an alkylene group having 2 to 12 carbon atoms:
[Chemical Formulae 4]
—C_{y}—C_{y} (4a)
—C_{y}—C_{y}—C_{y} (4b)
—C_{y}—Z—C_{y} (4c)
—C_{y}—Z—C_{y}—Z—C_{y} (4d)
—C_{y}—C≡C—C_{y} (4e )
—C_{y}—C≡C—C_{y}—C≡C—C_{y} (4f)
—C_{y}—Z—C_{y}—C≡C—C_{y} (4g )
where, in the general formulae (4a) to (4g): Z represents any one of —COO—, —OCO—, —CONH—, CON(alkyl)-, and —CH═N—; and C_{y}'s each independently represent any one of a phenyl ring, a naphthyl ring, a biphenyl ring, and a cyclohexyl ring which may each have at least one kind of substituent selected from F, CN, an alkoxy group, and an alkyl group.

2. A liquid crystal compound according to claim 1, comprising a chemical structure represented by any one of general formulae (5a) to (5f): 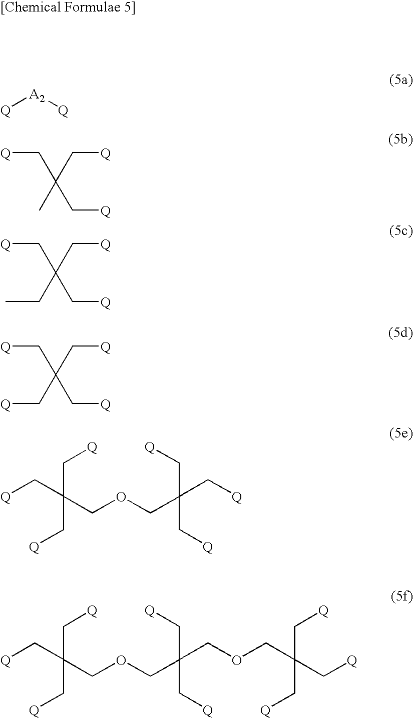
where, in the general formula (5a), A₂ represents an alkylene group having 2 to 12 carbon atoms, in which one —CH₂— or two or more nonadjacent —CH₂—'s present in the alkylene group may be substituted with —O—.

3. A liquid crystal compound according to claim 1, wherein J in the general formula (2) represents —H.

4. A liquid crystal compound according to claim 1, wherein Y in the general formula (2) represents —O—.

5. A liquid crystal compound according to claim 1, wherein X in the general formula (1) represents —CN.

6. A liquid crystal compound according to claim 1, wherein the liquid crystal compound comprises a cross-linkable liquid crystal compound.

7. An optical element, comprising the liquid crystal compound according to claim 1.

8. An optical element, comprising a cross-linked product formed by cross-linking the liquid crystal compound according to claim 6.

9. A polarizing plate, comprising the optical element according to claim 7.

10. An image display apparatus, comprising at least one polarizing plate according to claim 9.

11. An optical recording material, comprising the liquid crystal compound according to claim 1.
